(19) United States
(12) Patent Application Publication
(10) Pub. No.: US 2023/0014258 A1
(43) Pub. Date: Jan. 19, 2023

(54) DEVICE FOR MENISCAL REPAIR

(71) Applicant: LMF INDUSTRIES PTY LTD, Birtinya (AU)

(72) Inventors: Steven LAWRIE, Birtinya (AU); David Niles FRENCH, Wilston (AU); John Joseph MAGUIRE, Townsville (AU)

(73) Assignee: LMF INDUSTRIES PTY LTD, Birtinya (AU)

(21) Appl. No.: 17/757,660

(22) PCT Filed: Dec. 18, 2020
(86) PCT No.: PCT/AU2020/000138

§ 371 Date: Jun. 17, 2022

(30) Foreign Application Priority Data
Dec. 19, 2019 (AU) 2019904824

(51) Int. Cl. A61B 17/04; A61B 17/34

(52) U.S. Cl. CPC A61B 17/0401; A61B 17/0467; A61B 17/3421; A61B 2017/3445; A61B 2017/00464

(57) ABSTRACT

The present disclosure relates to a device for meniscal repair for use in areas of a human body where tissue can either be surgically reattached to bone or surgically repaired when a tear forms in the tissue. The device may take one of three forms; in a first form, the device comprises a handle and a deployment member permitting the deployment of anchors or sutures to the tissue requiring repair; subsequently the device may take a second form comprising the handle, still in place, and a cutter member for manipulating and cutting anchors and sutures to assist in repair of the tissue; in its third form, the device comprises the handle, still in place, and a fluid injection member for applying a fluid to aid in improving biological conditions for the tissue to heal.

### PRIORITY DOCUMENTS

The present application claims priority from Australian Provisional Patent Application No. 2019904824 titled “DEVICE FOR MENISCAL REPAIR” and filed on 19 Dec. 2019, the content of which is hereby incorporated by reference in its entirety.

### TECHNICAL FIELD

The present disclosure relates generally to the field of medical devices. In particular, the present disclosure comprises embodiments relating to a device for meniscal repair, the device being intended for use in areas in the body where tissue can be surgically reattached to bone or tissue or can be surgically repaired when a tear forms in the tissue.

### BACKGROUND

Soft tissue tears are a common injury to the cartilage that stabilises and cushions the joints known as the meniscus. The meniscus, which comprises a pair of C-shaped disks of cushioning material is located in the knee primarily between the femur and the tibia, and is particularly susceptible to injury. Meniscus injuries range from small to large tears that sometimes may heal on their own, but often require surgical intervention or repair.

A number of techniques have been developed by surgeons for repairing tissue, one of the most common techniques being arthroscopic or semi-open procedures which access the torn tissue through small openings (incisions) in the skin. This technique typically comprises the use of a first device creating at least one incision to a patient's tissue, for the delivery of a number of implants connected by a length of suture across the tear to close the tear and allow the tissue to heal. Often, the implementation of the implant and suture fails to provide sufficient repair to the torn tissue, and the repair may be improved by the creation of a more biologically advantageous condition for the tissue to heal. One such technique presently used for improving the biological condition for the tissue to heal, is to inject a biological agent by the use of a second device, such as a needle, to deliver any one of an adhesive, a local anaesthetic or an adjuvant agent to the torn tissue. Presently available devices and techniques often lead to an inaccurate delivery of the biological agent after the use of the first device, as the second device may not deliver the agent to the same location as the repair was made with the first device.

Inherent problems associated with these techniques for repairing tissues may include:
presently available devices typically only address a certain area of the torn tissue for repair, thus there is the requirement for surgeons to employ multiple devices to perform the repair thereby resulting in a reduced efficacy of the repair;
potential error in the biological agent not being applied by a second device to the desired location of the repair performed by the use of the first device; and
presently available devices used in tissue repair techniques are often difficult to use, not ergonomic, and demand that the surgeon performing the repair be familiar with the operation of a number of devices, this may lead to an ineffective repair or a failed repair to the tissue.

It is against this background and the problems and difficulties associated therewith, that the present disclosure has been developed.

Throughout this disclosure, the term “distal” with respect to a device for meniscal repair, a handle, an axial bore, a deployment member, a user operable actuator, a cannula, a through bore, a fluid injection member or a needle means the end of the device for meniscal repair, the handle, the axial bore, the deployment member, the user operable actuator, the cannula, the through bore, the fluid injection member or the needle further away from the direction of a patient, and the term “proximal” means a portion of the device for meniscal repair, the handle, the axial bore, the deployment member, the user operable actuator, the cannula, the through bore, the fluid injection member or the needle nearer to the direction of the patient.

Additionally, throughout this disclosure reference to a device for meniscal repair generally refers to a device for anchoring suture to a meniscus or a tissue. The term “tissue” is used throughout this disclosure to refer to any natural body tissue including, but not limited to, cartilage, skin, muscle, bone, tendon, ligament and organs, as well as to prosthetic materials that can be repaired or attached to natural body tissues with sutures and anchoring devices. The term “suture”, “implants”, “anchor”, “anchoring device”, and “suture anchor” may be understood to be fabricated substantially from surgical suture or any elongated, thread-like materials that can be used as medical devices (hereinafter referred to as either “suture” or “anchor”) referred to throughout this disclosure. The “suture” or “implant” can be non-absorbable, bioabsorbable, or partially bioabsorbable. Without deviation from the intent or scope of this disclosure, the “suture” or “implant” material may be mechanically or chemically modified, treated or coated to enhance lubricity, or its ability to repair tissue/meniscus, or other surgical purposes.

### SUMMARY

Embodiments of the present disclosure relate to a device for meniscal repair. The device for meniscal repair being intended for use in areas of a human body where a tissue may be surgically reattached to bone or can be surgically repaired when a tear forms in the tissue.

In certain embodiments, the device for meniscal repair comprises: a handle, wherein the handle comprises an axial bore extending between a first inlet and an outlet, and a first attachment portion; a cannula coupled to the outlet, wherein the cannula comprises a through bore cooperative with the axial bore of the handle; and wherein a portion of the axial bore at the first inlet of the handle is sized and shaped to form a socket, wherein the socket provides access to the axial bore of the handle and the through bore of the cannula.

According to a first aspect, there is provided a device for meniscal repair comprising: a handle comprising an axial bore extending between a first inlet and an outlet, and a first attachment portion; a cannula coupled to the outlet, wherein the cannula comprises a through bore cooperative with the axial bore of the handle; and wherein a portion of the axial bore at the first inlet of the handle is sized and shaped to form a socket, wherein the socket provides access to the axial bore of the handle and the through bore of the cannula.

In one form, the device for meniscal repair further comprises a deployment member comprising a first projection sized and shaped to be received within the socket of the handle.

In one form, the deployment member comprises an injector assembly configured to incrementally advance through the through bore of the cannula from a distal end to a proximal end of the cannula, wherein the injector assembly comprises one or more anchors, wherein the anchors are urged through the through bore in a predefined sequence to the proximal end, and a user operable actuator coupled to the injector assembly, wherein the user operable actuator provides an axial driving force to actuate the injector assembly.

In one form, the incremental advancement of the injector assembly through the through bore of the cannula provides a deployment of an anchor of the one or more anchors from the proximal end of the cannula.

In one form, the actuation of the user operable actuator to incrementally advance the injector assembly through the through bore of the cannula provides a tactile and audible indication of a deployment of the corresponding anchor of the one or more anchors.

In one form, the deployment member further comprises a deployment needle sized and shaped to be received within the through bore of the cannula, whereby the deployment needle deploys the anchor of the one or more anchors at the proximal end of the cannula.

In one form, the deployment needle comprises a flexible end which flexes to an angle of the proximal end of the cannula.

In one form, the deployment needle is manufactured of a transparent or a translucent material.

In one form, one of the handle or the deployment member further comprises an indicating means to provide a visual indication of a deployment of the corresponding anchor.

In one form, the deployment member further comprises a second attachment portion co-operable with the first attachment portion to form an attachment mechanism to effect securement of the deployment member to the handle.

In one form, one of the handle or the deployment member comprises a user operable release for the attachment mechanism.

In one form, the first or second attachment portions comprises a biasing means which biases the first or second attachment portions into a capture position with respect to the other attachment portion.

In one form, the user operable release acts against the biasing means, thereby releasing one of the first or second attachment portions from the capture position.

In one form, the device for meniscal repair further comprises a cutter member, wherein the cutter member comprises a second projection sized and shaped to be received within the socket of the handle, a pusher assembly configured to incrementally advance a cutting needle through the through bore of the cannula from a distal end to a proximal end of the cannula, wherein a cutting edge of the cutting needle is urged through the through bore in a predefined sequence to the proximal end, and a user operable actuator coupled to the pusher assembly, wherein the user operable actuator provides an axial driving force to actuate the pusher assembly.

In one form, the incremental advancement of the pusher assembly through the through bore of the cannula progressively protrudes the cutting edge of the cutting needle through the proximal end of the cannula.

In one form, the actuation of the user operable actuator to incrementally advance the pusher assembly through the through bore of the cannula provides a tactile and audible indication of the advancement of the cutting needle through the through bore of the cannula.

In one form, the cutting needle comprises a flexible end which flexes to an angle of the proximal end of the cannula.

In one form, the cutting needle is manufactured of a transparent or a translucent material.

In one form, one of the handle or the cutter member further comprises an indicating means to provide a visual indication of the incremental advancement of the cutting edge through the through bore of the cannula.

In one form, the cutter member further comprises a third attachment portion co-operable with the first attachment portion to form an attachment mechanism to effect securement of the cutter member to the handle.

In one form, one of the handle or the cutter member comprises a user operable release for the attachment mechanism.

In one form, at least one of the first or third attachment portions comprises a biasing means which biases the first or third attachment portions into a capture position with respect to the other attachment portion.

In one form, the user operable release acts against the biasing means, thereby releasing one of the first or third attachment portions from the capture position.

In one form, the cutting needle is sized and shaped to be received within the through bore of the cannula.

In one form, the device for meniscal repair further comprises: a fluid injection member for injecting a fluid, wherein the fluid injection member comprises a third projection sized and shaped to be received within the socket of the handle, a needle extending from a distal end to a proximal end of the fluid injection member, and a second inlet providing a fluid passageway extending between the second inlet and the proximal end of the needle.

In one form, the second inlet is sized and shaped to receive the fluid to be injected through the fluid passageway, whereby the fluid is expelled at the proximal end of the needle.

In one form, the fluid may be a biological agent or an adhesive or a local anaesthetic or an adjuvant agent used for meniscal repair.

In one form, the fluid injection member further comprises a fourth attachment portion co-operable with the first attachment portion to form an attachment mechanism to effect securement of the fluid injection member to the handle.

In one form, one of the handle or the fluid injection member comprises a user operable release for the attachment mechanism.

In one form, at least one of the first or fourth attachment portions comprises a biasing means which biases the first or fourth attachment portions into a capture position with respect to the other attachment portion.

In one form, the user operable release acts against the biasing means, thereby releasing one of the first or fourth portions from the capture position.

In one form, the needle is sized and shaped to be received within the through bore of the cannula.

In one form, a projected portion of the needle extends from the proximal end of the cannula.

In one form, the proximal end of the needle is flexible and flexes to an angle of the proximal end of the cannula.

In one form, a proximal end of the cannula is at an angle between 0° and 90°.

In one form, the meniscal repair device further comprises a depth gauge limiting the depth that the cannula may be inserted into a tissue.

In one form, the cannula is manufactured of a transparent or a translucent material.

According to a second aspect, there is provided a device for meniscal repair comprising: a handle comprising an axial bore extending between a first inlet and an outlet, and a first attachment portion; a cannula coupled to the outlet, wherein the cannula comprises a through bore cooperative with the axial bore of the handle, wherein a portion of the axial bore at the first inlet of the handle is sized and shaped to form a socket, wherein the socket provides access to the axial bore of the handle and the through bore of the cannula; and a deployment member comprising a first projection sized and shaped to be received within the socket of the handle, an injector assembly configured to incrementally advance through the through bore of the cannula from a distal end to a proximal end of the cannula, a user operable actuator coupled to the injector assembly, wherein the user operable actuator provides an axial driving force to actuate the injector assembly, and a second attachment portion co-operable with the first attachment portion to form an attachment mechanism to effect securement of the deployment member to the handle.

In one form, the injector assembly comprises one or more anchors, wherein the anchors are urged through the through bore in a predefined sequence to the proximal end.

In one form, the incremental advancement of the injector assembly through the through bore of the cannula provides a deployment of an anchor of the one or more anchors from the proximal end of the cannula.

In one form, the actuation of the user operable actuator to incrementally advance the injector assembly through the through bore of the cannula provides a tactile and audible indication of a deployment of the corresponding anchor of the one or more anchors.

In one form, the deployment member further comprises a deployment needle sized and shaped to be received within the through bore of the cannula, whereby the deployment needle deploys the anchor of the one or more anchors at the proximal end of the cannula.

In one form, one of the handle or the deployment member comprises a user operable release for the attachment mechanism.

In one form, at least one of the first or second attachment portions comprises a biasing means which biases the first or second attachment portions into a capture position with respect to the other attachment portion.

In one form, the user operable release acts against the biasing means, thereby releasing one of the first or second attachment portions from the capture position.

According to a third aspect, there is provided a device for meniscal repair comprising: a handle comprising an axial bore extending between a first inlet and an outlet, and a first attachment portion; a cannula coupled to the outlet, wherein the cannula comprises a through bore cooperative with the axial bore of the handle, wherein a portion of the axial bore at the first inlet of the handle is sized and shaped to form a socket, wherein the socket provides access to the axial bore of the handle and the through bore of the cannula; and a cutter member, wherein the cutter member comprises a second projection sized and shaped to be received within the socket of the handle, a pusher assembly configured to incrementally advance a cutting needle through the through bore of the cannula from a distal end to a proximal end of the cannula, wherein a cutting edge of the cutting needle is urged through the through bore in a predefined sequence to the proximal end, a user operable actuator coupled to the pusher assembly, wherein the user operable actuator provides an axial driving force to actuate the pusher assembly, and a third attachment portion co-operable with the first attachment portion to form an attachment mechanism to effect securement of the cutter member to the handle.

In one form, the incremental advancement of the pusher assembly through the through bore of the cannula progressively protrudes the cutting edge of the cutting needle through the proximal end of the cannula.

In one form, the actuation of the user operable actuator to incrementally advance the pusher assembly through the through bore of the cannula provides a tactile and audible indication of the advancement of the cutting needle through the through bore of the cannula.

In one form, one of the handle or the cutter member further comprises an indicating means to provide a visual indication of the incremental advancement of the cutting edge through the through bore of the cannula.

In one form, one of the handle or the cutter member comprises a user operable release for the attachment mechanism.

In one form, the first or third attachment portions comprises a biasing means which biases the first or third attachment portions into a capture position with respect to the other attachment portion.

In one form, the user operable release acts against the biasing means, thereby releasing one of the first or third attachment portions from the capture position.

In one form, the cutting needle is sized and shaped to be received within the through bore of the cannula.

According to a fourth aspect, there is provided a device for meniscal repair comprising: a handle comprising an axial bore extending between a first inlet and an outlet, and a first attachment portion; a cannula coupled to the outlet, wherein the cannula comprises a through bore cooperative with the axial bore of the handle, wherein a portion of the axial bore at the first inlet of the handle is sized and shaped to form a socket, wherein the socket provides access to the axial bore of the handle and the through bore of the cannula; and comprising a fluid injection member for injecting a fluid, wherein the fluid injection member comprises a third projection sized and shaped to be received within the socket of the handle, a needle extending from a distal end to a proximal end of the fluid injection member, a second inlet providing a fluid passageway extending between the second inlet to the proximal end of the needle, and comprises a fourth attachment portion co-operable with the first attachment portion to form an attachment mechanism to effect securement of the fluid injection member to the handle.

In one form, the second inlet is sized and shaped to receive the fluid to be injected through the fluid passageway, whereby the fluid is expelled at the proximal end of the needle.

In one form, the fluid may be a biological agent or an adhesive or a local anaesthetic or an adjuvant agent used for meniscal repair.

In one form, one of the handle or the fluid injection member comprises a user operable release for the attachment mechanism.

In one form, the first or fourth attachment portions comprises a biasing means which biases the first or fourth attachment portions into a capture position with respect to the other attachment portion.

In one form, the user operable release acts against the biasing means, thereby releasing one of the first or fourth portions from the capture position.

In one form, the needle is sized and shaped to be received within the through bore of the cannula.

### BRIEF DESCRIPTION OF DRAWINGS

Embodiments of the present disclosure will be discussed with reference to the accompanying drawings wherein:

FIG. 1 is a side view of an embodiment of a device for meniscal repair comprising a deployment member;

FIG. 2 is a schematic sectional view of the device for meniscal repair comprising the deployment member taken along line A-A in FIG. 1;

FIG. 3 is a side view of an embodiment of a handle for the device for meniscal repair of any one of FIG. 1 or 2, the multiple illustrations of the handle include additional embodiments of the handle with various angles at which a cannula of the handle may be bent;

FIG. 4 is a schematic sectional view of the handle taken along line B-B in FIG. 3;

FIG. 5 is a side view of an alternative embodiment of a handle for the device for meniscal repair;

FIG. 6 is a schematic sectional view of the handle taken along line C-C in FIG. 5;

FIG. 7 is a side view of an embodiment of the deployment member of FIGS. 1 and 2;

FIG. 8 is a schematic sectional view of the deployment member taken along line D-D in FIG. 7;

FIG. 9 is a schematic sectional view illustrating Detail X of the deployment member of FIG. 8;

FIG. 10 is a side view of an alternate embodiment of a device for meniscal repair comprising a cutter member;

FIG. 11 is a schematic sectional view of the device for meniscal repair comprising the cutter member taken along line E-E in FIG. 11;

FIG. 12 is a side view of an embodiment of the cutter member of FIGS. 10 and 11;

FIG. 13 is a schematic sectional view of the cutter member taken along line F-F of FIG. 12;

FIG. 14 is a schematic sectional view illustrating Detail Y of the cutter member of FIG. 13;

FIG. 15 is a side view of an alternate embodiment of a device for meniscal repair comprising a fluid injection member for injecting a fluid;

FIG. 16 is a schematic sectional view of the device for meniscal repair comprising the fluid injection member taken along line G-G in FIG. 15;

FIG. 17 is a side view of an embodiment of the injection member of FIGS. 15 and 16;

FIG. 18 is a schematic sectional view of the injection member taken along line H-H of FIG. 17;

FIG. 19 is a top view of an alternative embodiment of a device for meniscal repair comprising an alternative fluid injection member for injecting a fluid;

FIG. 20 is a schematic sectional view of the alternative device for meniscal repair taken along line I-I of FIG. 19;

FIG. 21 is a side view of the alternative device for meniscal repair of FIGS. 19 and 20;

FIG. 22 is a schematic sectional view of the alternative device for meniscal repair taken along line J-J of FIG. 21;

FIG. 23 is a side view of an embodiment of a device for meniscal repair comprising an alternate deployment member and the handle of FIGS. 5 and 6;

FIG. 24 is a schematic sectional view of the device for meniscal repair taken along line K-K of FIG. 23;

FIG. 25 is a side view of the alternate deployment member of FIGS. 23 and 24;

FIG. 26 is a schematic sectional view of the alternate deployment member taken along line L-L of FIG. 25;

FIG. 27 is a top view of an embodiment of a further alternate deployment member for a device for meniscal repair;

FIG. 28 is a schematic sectional view of the alternate deployment member taken along line M-M of FIG. 27;

FIG. 29 is a side view of the alternate deployment member of FIGS. 27 and 28;

FIG. 30 is a schematic sectional view of the alternate deployment member taken along line N-N of FIG. 29;

FIG. 31 is a top view of an embodiment of an alternate cutter member for a meniscal repair device;

FIG. 32 is a schematic sectional view of the alternate cutter member taken along line O-O of FIG. 31;

FIG. 33 is a side view of the alternate cutter member of FIGS. 31 and 32; and

FIG. 34 is a schematic sectional view of the alternate cutter member taken along line P-P of FIG. 33.

In the following description, like reference characters designate like or corresponding parts throughout the figures.

### DESCRIPTION OF EMBODIMENTS

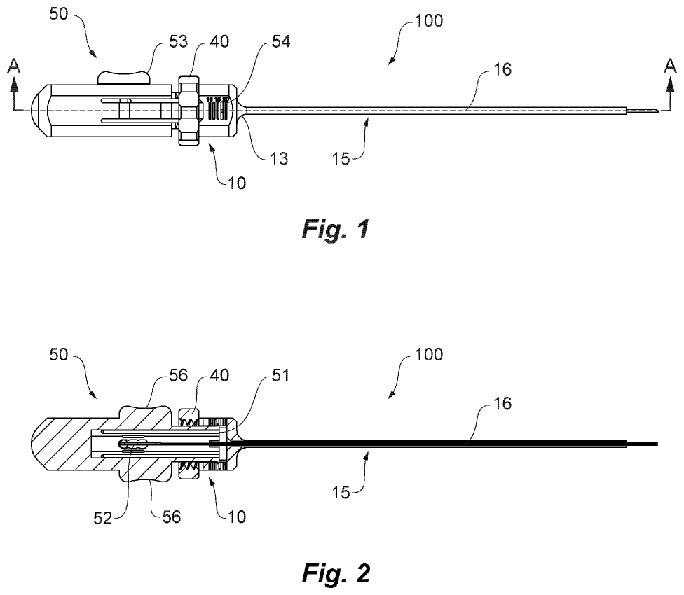

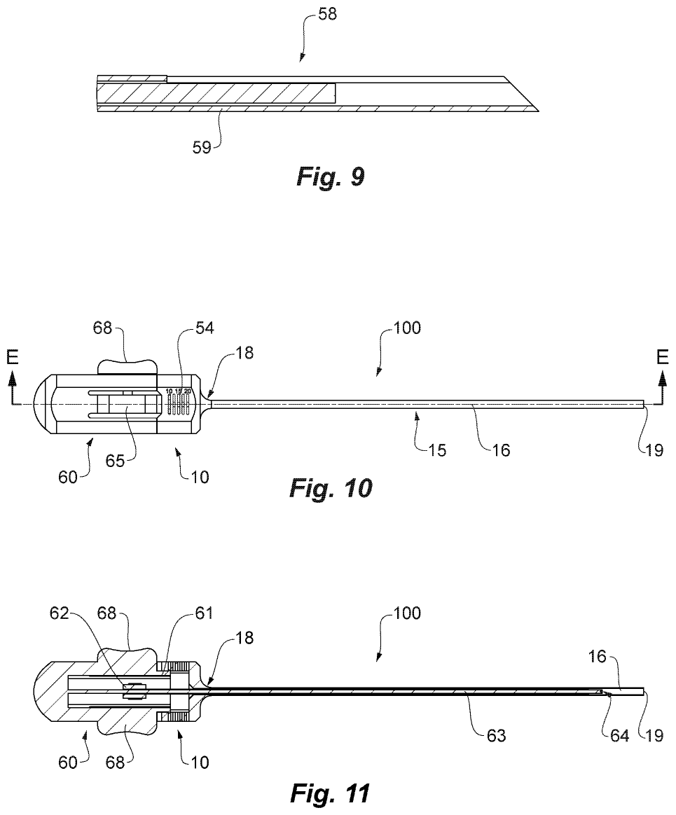

Referring to any one of FIGS. 1 to 34, there is illustrated a device for meniscal repair (100) according to a number of embodiments. The device for meniscal repair (100) is intended for use in areas in the body where a tissue may be surgically reattached to bone or can be surgically repaired when a tear forms in the tissue. The device for meniscal repair (100) is a particularly designed medical device for use in repairing one or more tears in a meniscus (a tissue comprising a pair of C-shaped disks of cushioning material located in the knee primarily between the femur and the tibia). Additionally, the device for meniscal repair (100) is particularly designed such that certain embodiments of the device (100) may have applications in “inside out” meniscal repair, and other embodiments may find use in “all inside” meniscal repair.

The device for meniscal repair (100) may comprise a handle (10). The handle (10) may comprise an axial bore (11), the axial bore (11) extending between a first inlet (12) and an outlet (13) of the handle (10). The device for meniscal repair (100) may also comprise a first attachment portion (17) and a cannula (15). The cannula (15) may be coupled to the outlet (13) of the handle (10), the cannula (15) may comprise a through bore (16) cooperative with the axial bore (11) of the handle (10). The axial bore (11) of the handle (10) may comprise a portion at the outlet (13) sized and shaped to form a socket (14). The socket (14) may provide access to the axial bore (11) of the handle (10) and the through bore (16) of the cannula (15).

Referring to any one of FIGS. 1 to 9, in one embodiment, the device for meniscal repair (100) may further comprise a deployment member (50). The deployment member (50) may comprise a first projection (51), whereby the first projection (51) is sized and shaped to be received in the socket (14) of the handle (10). In this way, it will be appreciated, that the socket (14) of the handle (10) may house the first projection (51) of the deployment member (50). Thereby, in use, the handle (10) and the deployment member (50) cooperate so as to form a first embodiment of the device for meniscal repair (100).

In this particular embodiment, the deployment member (50) may further comprise an injector assembly (52). Whereby, the injector assembly (52) is configured to incrementally advance through the through bore (16) of the cannula (15) from a distal end (18) to a proximal end (19) of the cannula (15). In this way, the injector assembly (52) may have an initial position at the distal end (18) of the cannula (15) and be configured to incrementally advance through the cannula (15) to a final position at the proximal end (19) of cannula (15).

In this particular embodiment, the injector assembly (52) may comprise one or more anchors, whereby the one or more anchors are urged through the through bore (16) of the cannula (15) from the distal end (18) in a predefined sequence to the proximal end (19). The predefined sequence may correspond to the configuration of the injector assembly (52) to incrementally advance through the through bore (16) of the cannula (15) from its initial position at the distal end (18) of the to the final position at the proximal end (19) of the cannula (15).

In one embodiment, the deployment member (50) may further comprise a user operable actuator (53). The user operable actuator (53) may be coupled to the injector assembly (52), whereby the user operable actuator (53) may provide an axial driving force to actuate the injector assembly (52). In this way, the user operable actuator (53) may provide the axial driving force to actuate the predefined sequence that the one or more anchors are urged through the through bore (16) of the cannula (15). The user operable actuator (53) may be sized and shaped to be comfortable and ergonomically located for a user to hold and operate the device for meniscal repair (100) over an extended period of time. As illustrated in any one of FIGS. 1, 2 and 7 and 8, a handling portion of the deployment member (50) may take a shape and size to comfortably and ergonomically fit into the palm of the user to hold and operate the device for meniscal repair (100). It will be appreciated that other sizes and shapes are envisaged for the user operable actuator (53), suitable for various means of operation to deliver the axial driving force to drive the predefined sequence corresponding to the configuration of the injector assembly (52) to incrementally advance through the through bore (16) of the cannula (15).

In this particular embodiment, the incremental advancement of the injector assembly (52) through the through bore (16) of the cannula (15) from its initial position at the distal end (18) of the cannula (15) to the final position at the proximal end (19), may provide for a deployment of one of the anchors of the one or more anchors from the proximal end (19) of the cannula (15). In this way, it will be understood that the arrangement of the deployment member (50) permits that the use of the user operable actuator (53) delivers the axial driving force required for the injector assembly (52) to commence its predefined sequence to urge the one or more anchors through the through bore (16) of the cannula (15) of the handle (10), and ultimately result in the deployment of one of the anchors of the one or more anchors at the proximal end (19) of the cannula (15). It will also be appreciated that the device for meniscal repair (100) will typically be configured so as to deliver one of the anchors of the one or more anchors, but the device (100) may also be configured to deliver more than one of the anchors of the one or more anchors at the proximal end (19) of the cannula (15).

In one embodiment, the actuation of the user operable actuator (53) to incrementally advance the injector assembly (52) through the through bore (16) of the cannula (15) may provide a tactile and audible indication of the deployment of the corresponding anchor of the one or more anchors. In this way, the user is provided with an indication via the tactile and audible response of the user operable actuator (53) is actuated, and that one of the anchors of the one or more anchors have been deployed at the proximal end (19) of the cannula (15).

In any one of the above embodiments, the deployment member (50) may further comprise a deployment needle (58). The deployment needle (58) may be sized and shaped to be received within the through bore (16) of the cannula (15) of the handle (10), whereby the deployment needle (58) deploys the anchor of the one or more anchors at the proximal end (19) of the cannula (15). In this way, the first projection (51) of the deployment member (50) when received within the socket (14) of the handle (10), may also cause the through bore (16) of the cannula (15) to receive the deployment needle (58) of the deployment member (50), thereby forming the first embodiment of the device for meniscal repair (100). In this particular embodiment, it will be appreciated that the deployment needle (58) may comprise any number of anchors of the one or more anchors (not shown) that may be deployed in the predetermined sequence.

In one embodiment, the deployment needle (58) may further comprise a flexible end (59). Whereby the flexible end (59) may flex to an angle of the proximal end (19) of the cannula (15). In this way, when the proximal end (19) of the cannula (15) is set at a particular angle, the flexible end (59) of the deployment needle (58) may flex to said particular angle. It will be appreciated that the flexible end (59) may be sized and shaped to be received within the through bore (16), and project from the proximal end (19) of the cannula (15), whereby it may also comprise any number of anchors of the one or more anchors (not shown) that may be deployed in the predetermined sequence.

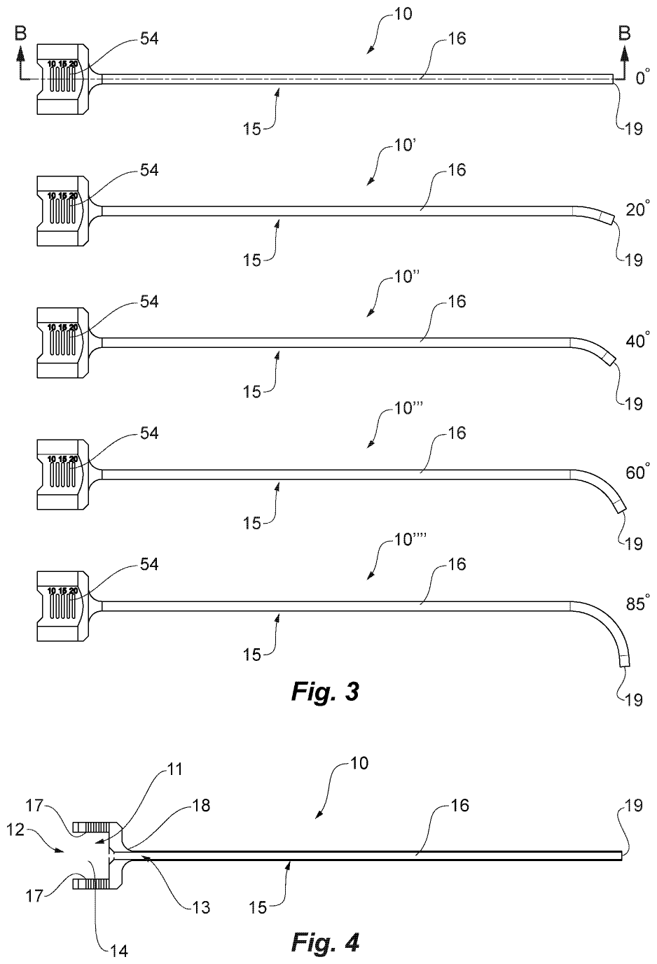

In any one of the above embodiments, the handle (10) of the first embodiment of the device for meniscal repair (100) comprising the deployment member (50), may further comprise an indicating means (54) to provide a visual indication of a deployment of the corresponding anchor. Particularly with reference to any one of FIG. 1 or 3, it is illustrated that the indicating means (54) may be positioned on the handle (10) so as to provide the user with the visual indication.

In this particular embodiment, the indicating means (54) may comprise one or more indicia to provide the visual indication of the deployment of the anchor of the one or more anchors. In this way, the indicating means (54) may include a number of indicia corresponding to the number of anchors of the one or more anchors that may be deployed. It will be appreciated that further embodiments of the indicating means (54) are envisaged beyond those discussed, such as any indicating means (54) that may in use provide the user with a visual indication of the deployment of the anchor of the one or more anchors.

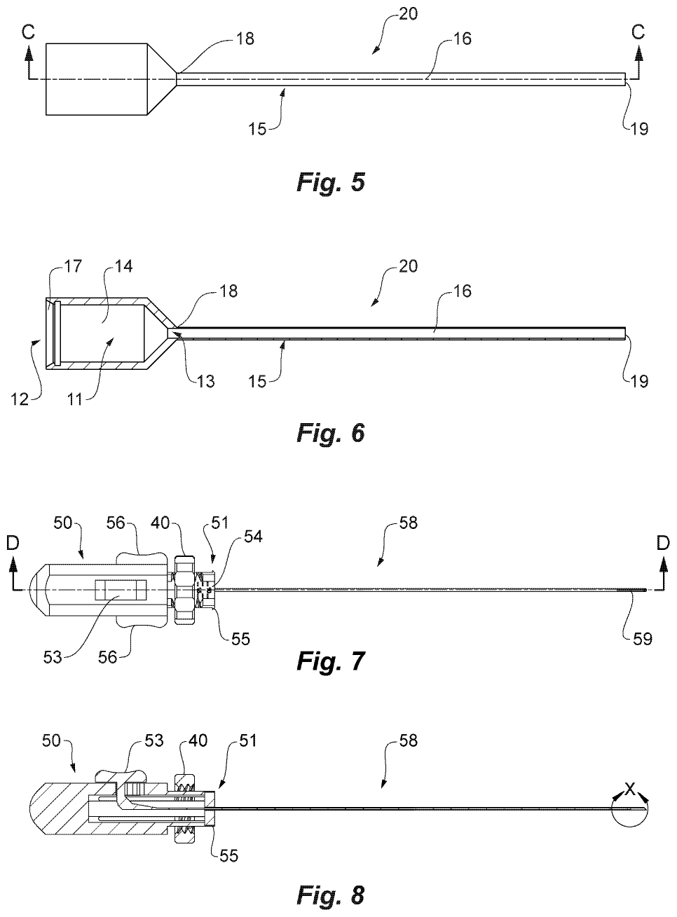

In an alternate embodiment, referring now to any one of FIG. 5 or 6, an alternate handle (20) is illustrated. In this alternate embodiment, the alternate handle (20) does not comprise the indicating means (54) of the handle (10), however may comprise similar features such as the axial bore (11) extending between the first inlet (12) and the outlet (13) of the handle (20). Also similarly, the alternate handle (20) may also comprise the first attachment portion (17) and the cannula (15), where the cannula (15) is coupled to the outlet (13) of the alternate handle (20). The cannula (15) may comprise the through bore (16) cooperative with the axial bore (11) of the alternate handle (20). The axial bore (11) of the alternate handle (20) may comprise a portion at the outlet (13) sized and shaped to form the socket (14). The socket (14) may provide access to the axial bore (11) of the handle (10) and the through bore (16) of the cannula (15). In this alternate embodiment, the alternate handle (20) is of a simpler construction and use than the handle (10). The alternate handle (20) may be constructed of a transparent or translucent material, advantageously, in this way the visual indication of the deployment of the anchor of the one or more anchors is simply possible by viewing through the transparent or translucent alternative handle (20).

It will be appreciated that the above embodiment of the alternate handle (20) may function in all other aspects similar to that of the handle (10), where like feature references in FIG. 5 or 6 concord with like feature references in FIGS. 1 to 4 of the handle (10). Additionally, in the following disclosure, it will be appreciated that the features discussed below may be applicable, in certain embodiments, to either handle (10 or 20).

In one embodiment, referring now to any one of FIGS. 1, 2, 7 and 8, the deployment member (50) may further comprise a depth setting wheel (40). The depth setting wheel (40) being configured so as to position the flexible end (59) of the deployment needle (58) relative to the proximal end (19) of the cannula (15). In this way, the depth setting wheel (40) may provide the user with control to advantageously accurately position the flexible end (59) of the deployment needle (58) relative to the proximal end (19) of the cannula (15).

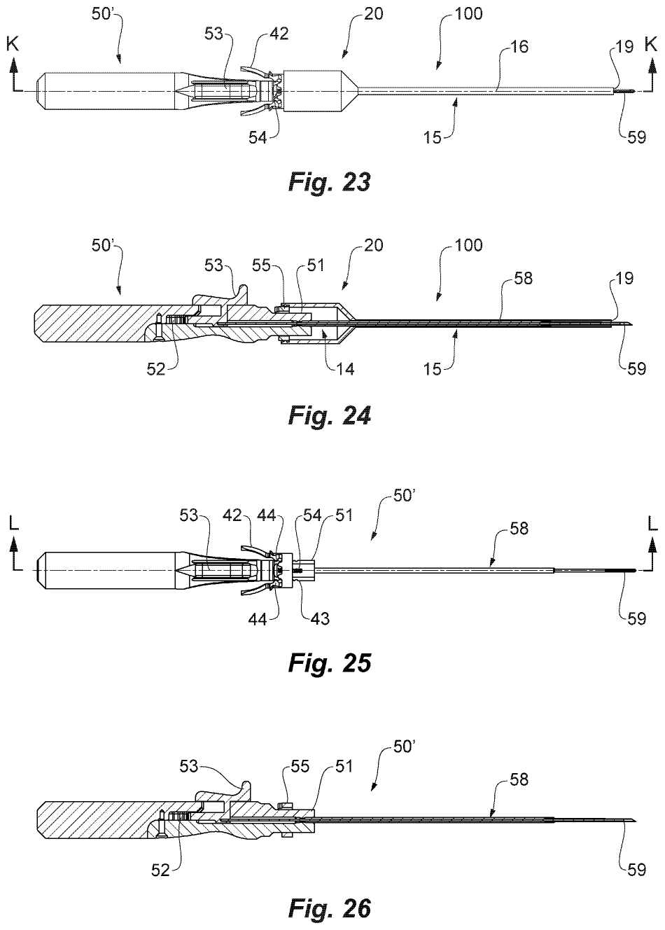

In an alternate embodiment to the above, referring now to any one of FIGS. 23 to 26, an alternate deployment member (50′) is illustrated with a depth setting mechanism (42). In this alternate embodiment, the depth setting mechanism (42) may be proximal to the first projection (51) of the alternate deployment member (50′) and is configured so as to position the flexible end (59) of the deployment needle (58) relative to the proximal end (19) of the cannula (15) of the alternate handle (20).

Additionally, in this alternate embodiment, the first projection (51) of the alternate deployment member (50′) may comprise one or more keepers (43) along a length of the first projection (51), the length extending between a proximal end and a distal end of the first projection (51). Whereby, each of the keepers (43) along the length of the first projection (51) correspond to a position of the flexible end (59) of the deployment needle (58) relative to the proximal end (19) of the cannula (15) of the alternate handle (20). The first projection (51) of the alternate deployment member (50′) may further comprise the indicating means (54) along the length of the first projection, and similarly corresponding to the position of the flexible end (59) of the deployment needle (58) relative to the proximal end (19) of the cannula (15) of the alternate handle (20). In this way, each of the keepers (43) correspond to the indicating means (54), such that at each of the keepers (43) position the flexible end (59) relative to the proximal end (19) so as to allow the user to visually identify via the one or more indicia of the indicating means (54) said position.

Furthermore, the depth setting mechanism (42) may comprise one or more ribs (44), such that the one or more ribs (44) concord to the one or more keepers (43) along the length of the first projection (51). The depth setting mechanism (42) being particularly designed so as to bias the one or more ribs (44) into any one of the one or more keepers (43), such that by cooperating the one or more ribs (44) with one or more keepers (43) along the length of the first projection (51), in use, is able to position the flexible end (59) of the deployment needle (58) relative to the proximal end (19) of the cannula (15) of the alternate handle (20).

It will be appreciated that the above alternate deployment member (50′) may function in all aspects similar to that of the deployment member (50), where like feature references in FIGS. 23 to 26 concord with like feature references in FIGS. 1, 2, 7 and 8 of the deployment member (50). Additionally, in the following disclosure, it will be appreciated that the features discussed below may be applicable, in certain embodiments, to either deployment member (50 or 50′).

In one embodiment, the user operable actuator (53) may further comprise a lock mechanism (not shown) to prevent the user from incrementally advancing the injector assembly (52) through the through bore (16) of the cannula (15). Thereby, the lock mechanism of the user operable actuator (53) may prevent the accidental deployment of any one of the one or more anchors, and the actuation of the user operable actuator (53).

In one embodiment, the user operable actuator (53) may comprise a first handling portion. The first handling portion may provide the user with an ergonomically positioned handle to permit the actuation of the user operable actuator (53) to incrementally advance the injector assembly (52) through the through bore (16) of the cannula (15). In this particular embodiment, the first handling portion may comprise a fluted surface (not shown), whereby the fluted surface permits the user to easily handle and grip the first handling portion to actuate the user operable actuator (53).

In one embodiment, the deployment member (50 or 50′) may further comprise a second attachment portion (55), co-operable with the first attachment portion (17) of the handle (10) so as to form an attachment mechanism (not shown) to effect securement of the deployment member (50 or 50′) to the handle (10 or 20). In this way, the deployment member (50 or 50′) is secured to the handle (10 or 20) so as to form the first embodiment of the device for meniscal repair (100).

In one embodiment, one of the handle (10 or 20) or the deployment member (50 or 50′) may comprise a user operable release (56) for the attachment mechanism.

In this particular embodiment, one of either the first attachment portion (17) of the handle (10) or the second attachment portion (55) of the deployment member (50) may comprise a biasing means (not shown). The biasing means may bias either one of the first (17) or second (55) attachment portions into a capture position with respect to the other attachment portion. In this way, the capture position may represent a locked position in which the deployment member (50) is secured to the handle (10), thereby forming the first embodiment of the device for meniscal repair (100).

In this particular embodiment, the user operable release may act against the biasing means, thereby releasing one of the first (17) or second (55) attachment portions from the capture position. The biasing means may be manufactured of a resilient material which biases one of the first (17) or second (55) attachment portions into the capture position with respect to the other attachment portion. The biasing means may comprise a number of features such as a spring (not shown) which may be used to bias one of the first (17) or second (55) attachment portions into the capture position with respect to the other attachment portion. Alternate biasing means beyond those described in the embodiments of this disclosure are also envisaged, it will be appreciated by those skilled in the art that the biasing means coupled with the first (17) and second (55) attachment portions are designed so as to provide a releasable mechanism that effects securement of the deployment member (50) to the handle (10), thereby forming the first embodiment of the device for meniscal repair (100). It will be further appreciated that the releasable mechanism that effects this securement, may be known to provide for quick coupling and uncoupling of the deployment member (50) from the handle (10), whereby the handle (10) may be further used to receive another member (than the deployment member (50)) within the socket (14).

In an alternative embodiment, the user operable release (56) may take the form of a button (not shown) or a trigger (not shown), and may be comprised on either one of the handle (10) or the deployment member (50) of the device for meniscal repair (100). Illustrated in any one of FIG. 2 or 7, the user operable release (56) may take the form of two or more buttons (56) that must be actuated simultaneously in order to provide the releasable mechanism that effects securement of the deployment member (50) to the handle (10). It will be appreciated that the user operable release (56) may take further alternate forms beyond those described in these embodiments. In this way, the user operable release (56) provides the releasable mechanism that effects securement of the deployment member (50) to the handle (10).

In another alternative embodiment, one of the first (17) or second (55) attachment portions may be a cantilever snap, and the other may be a keeper for the cantilever snap. In this particular embodiment, the user operable release (56) acts against the cantilever snap, thereby releasing it from the keeper. In this way, the user operable release (56) provides the releasable mechanism that effects securement of the deployment member (50) to the handle (10).

In another alternative embodiment, one of the first (17) or second (55) attachment portions may comprise one or more ribs, and the other may comprise one or more keepers (not shown) corresponding to the one or more ribs. In this particular embodiment, the user operable release (56) may act against the attachment mechanism formed by the cooperation of the one or more ribs with the one or more keepers to provide the releasable mechanism that effects securement of the deployment member (50 or 50′) to the handle (10 or 20).

In another alternative embodiment, referring now to any one of FIGS. 23 to 26, one of the first attachment portion (17) of the alternate handle (20) or second attachment portion (55) of the alternate deployment member (50′) may comprise male threads, and the other may comprise female threads corresponding to and able to threadably engage the male threads. In this particular embodiment, it will be appreciated that the attachment mechanism is formed by the cooperation of the male and female threads to secure the alternate deployment member (50′) to the alternate handle (20) to thereby form an embodiment of the device for meniscal repair (100). It will be further appreciated that in this alternate embodiment, the device for meniscal repair (100) may be designed without the user operable release.

In a further alternate embodiment, it will be appreciated that the first embodiment of the device for meniscal repair (100) comprising the deployment member (50 or 50′) may be designed without the user operable release, or the biasing means. In this embodiment, the deployment member (50 or 50′) may simply be secured to the handle (10 or 20) by virtue of the first projection (51) being sized and shaped to be received within the socket (14). In this way, a person skilled in the art will appreciate that the deployment member (50 or 50′) may be removably secured to the handle (10 or 20).

In any one of the above embodiments, the device for meniscal repair (100) comprising the deployment member (50 or 50′) comprising a length of suture and the one or more anchors which may be utilised for anchoring of the suture directly to torn tissue/meniscus, it will be appreciated that the anchors may be used to anchor suture to an existing implant (not shown) already utilised for repair to the torn tissue/meniscus. The existing implant can be any variety of implant types including but not limited to a suture anchor, a cannulated screw or rivet, and another implanted object such as a bone plate. Fixation of the existing implant can be by any known means including but not limited to interference, toggling, screw threads expandable or extendable members, biological agents, adhesives and cements.

In any one of the above embodiments, the device for meniscal repair (100) comprising the deployment member (50 or 50′) may be used for the repair of soft tissues, such as a meniscus, that may have been torn. The repair of the tissue/meniscus may be carried out by known arthroscopic or semi-open procedures well known, utilising the embodiments of the device for meniscal repair (100) by the creation of at least one incision to a tissue created by insertion of the proximal end (19) of the cannula (15) at the desired incision location. Following the creation of the incision, the cannula (15) of the device for meniscal repair (100) may be surgically directed toward the location repair is to be carried out, and the user of the device (100) may actuate the user operable actuator (53) to incrementally advance the injector assembly (52) through the through bore (16) of the cannula (15) to deliver the desired number of one or more anchors required to repair the tear at the tissue/meniscus, whilst receiving confirmation of the deployment of the anchor of the one or more anchors at the tear via the tactile and audible response, and a visual indication of the deployment via the indicating means (54). It will be appreciated, that the above embodiments of the device for meniscal repair (100) represent the first embodiment of the device (100) as illustrated by any one of FIGS. 1, 2, 23 and 24.

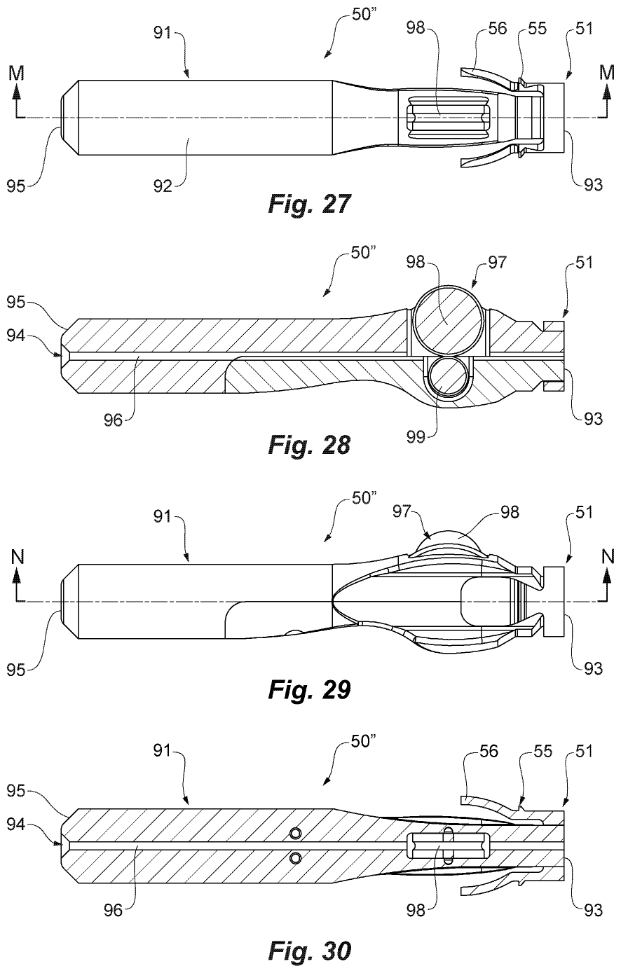

In a further embodiment, either of the deployment members (50 or 50′) may take an alternate form as in any one of FIGS. 27 to 30, illustrating a further deployment member (50″). In this particular embodiment, the deployment member (50″) may comprise like features referenced with the same numerals as those of deployment members (50 or 50′), accordingly for these like features, it will be appreciated that the function of these will not be discussed in further detail as they function similar as to what has already been disclosed. The deployment member (50″) comprises the first projection (51) which is sized and shaped to be received in the socket (14) of the alternate handle (20). In this way, it will be appreciated that the socket (14) of the handle (20) may house the first projection (51) of the deployment member (50″). Thereby, in use, the handle (20) and the deployment member (50″) cooperate so as to form the first embodiment of the device for meniscal repair (100).

In the above embodiment, the deployment member (50″) comprises a body (91) comprising a handle portion (92) extending longitudinally away from the first projection (51), whereby the first projection is at a proximal end (93) of the deployment member (50″) and a needle inlet (94) is disposed on the handle portion (92) at a distal end (95) of the deployment member (50″). The needle inlet (94) provides a needle passageway (96) extending between the needle inlet (94) at the distal end (95) to the proximal end (93) of the deployment member (50″). In this embodiment, the needle passageway (96) is co-operable with the through bore (16) of the cannula (15) of the alternate handle (20) so as to permit a needle (not shown) inserted at the needle inlet (94) to travel from the distal end (95) through the needle passageway (96) and subsequently the through bore (16) of the cannula (15) to be expelled through the proximal end (19) of the cannula (15).

The deployment member (50″) of the above embodiment may further comprise a user operable pusher assembly (97). The pusher assembly (97) may be coupled to the needle passageway (96), and thus the needle (not shown) travels through the needle passageway (96) between the distal (95) and proximal (93) ends of the deployment member (50″). The pusher assembly (97) comprising an upper wheel (98) and a lower wheel (99), whereby the needle passageway (96) and the needle extend between the upper (98) and lower (99) wheels, that in use, rotation of the upper wheel (98) provides an axial driving force to the needle within the needle passageway (96) to drive it either toward the proximal end (93) and subsequently through bore (16) of the cannula (15) to be expelled through the proximal end (19) of the cannula (15), or the driving force derived from the upper wheel (98) may drive the needle in the needle passageway (96) toward the distal end (95) and away from the proximal end (19) of the cannula (15). It will be appreciated that the upper (98) and lower (99) wheels are proximal to one another, such that a space between the upper (98) and lower (99) wheels is cable of gripping and driving the needle within the needle passageway (96). It will also be appreciated that the upper wheel (98) may be so positioned on the body (91) so as to be manipulable by the user to drive the needle in the needle passageway (96), and the lower wheel (99) may be housed within an internal portion of the body (91) and below the needle passageway (96). It will be further appreciated that the user operable pusher assembly (97) of the deployment member (50″) advantageously allows the user to drive the needle without predefined increments in either the proximal or distal direction. Advantageously, this provides the user with greater control of the needle required for meniscal repair.

Still referring to the above embodiment, the deployment member (50″) comprises the second attachment portion (55) which, in use, operates as a cantilever snap, and the first attachment portion (17) of the alternate handle (20) may be a keeper for said cantilever snap. In this way, the user operable release (56) acts against the cantilever snap, thereby releasing it from the keeper. In this way, the user operable release (56) provides the releasable mechanism that effects securement of the deployment member (50″) to the alternate handle (20).

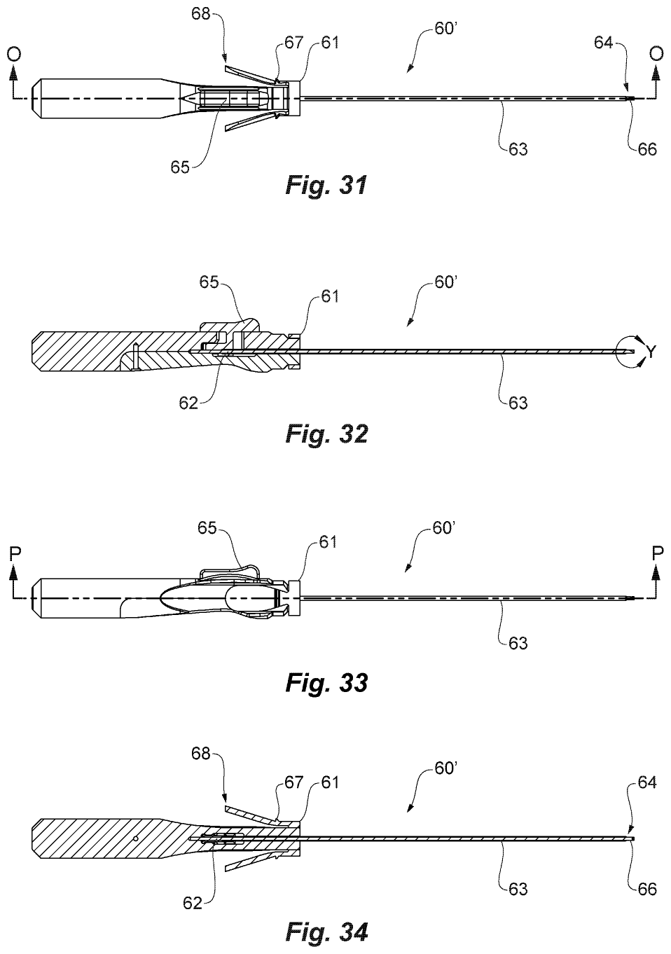

In an alternative embodiment, the device for meniscal repair (100) may comprise either a cutter member (60) (illustrated in FIGS. 10 to 14) or an alternate cutter member (60′) (illustrated in FIGS. 31 to 34). In the feature descriptions that follow, it will be appreciated that like feature references between FIGS. 10 to 14 illustrating the cutter member (60) and FIGS. 31 to 34 illustrating the alternate cutter member (60′) refer to corresponding parts, and may be applicable to either cutter member (60 or 60′).

In either embodiment, the cutter members (60 or 60′) may comprise a second projection (61), whereby the second projection may be sized and shaped to be received within the socket (14) of the handle (10) or the alternate handle (20). In this way, it will be appreciated that the socket (14) of the handle (10 or 20) may house the second projection (61) of the cutter member (60 or 60′). Thereby, in use, the handle (10 or 20) and the cutter member (60 or 60′) cooperate so as to form a second embodiment of the device for meniscal repair (100).

In one embodiment, the cutter member (60 or 60′) may further comprise a pusher assembly (62). The pusher assembly (62) is configured to incrementally advance a cutting needle (63) through the through bore (16) of the cannula (15) from the distal end (18) to the proximal end (19) of the cannula (15). In this way, the pusher assembly (62) may have an initial position at the distal end (18) of the cannula (15) and be configured to incrementally advance through the cannula (15) to a final position at the proximal end (19) of the cannula (15).

In this particular embodiment, the cutting needle (63) may comprise a cutting edge (64) that may be urged through the through bore (16) in the predefined sequence to the proximal end (19) of the cannula (15). The predefined sequence may correspond to the configuration of the pusher assembly (62) to incrementally advance through the through bore (16) of the cannula (15) from its initial position at the distal end (18) of the to the final position at the proximal end (19) of the cannula (15).

Additionally in this particular embodiment, the incremental advancement of the pusher assembly (62) through the through bore (16) of the cannula (15) progressively protrudes the cutting edge (64) of the cutting needle (63) through the proximal end (19) of the cannula (15). The cutting edge (64) may be sufficiently sharp so as to be able to cut a suture. In this way, the cutting edge (64) may be utilised to cut excess suture that may extend beyond that required for the repair of the torn tissue/meniscus. In an alternative, the cutting edge (64) may also be utilised to place, tension and slacken suture used in the repair of the torn tissue/meniscus. In a further alternative, the cutting edge (64) may be utilised to manipulate a length of suture for use in the repair of the torn tissue/meniscus. It will be appreciated by those skilled in the art, that further uses of the cutting edge (64) are envisaged, particularly those that enable the cutting edge (64) to be used by the user of the device for meniscal repair (100) comprising the cutter member (60 or 60′) to manipulate, cut, tension, slacken or remove suture from use in the repair of the torn meniscus/tissue.

In one embodiment, the cutter member (60 or 60′) may further comprise a user operable actuator (65). The user operable actuator (65) may be coupled to the pusher assembly (62), whereby the user operable actuator (65) provides an axial driving force to actuate the pusher assembly (62). In this way, the user operable actuator (65) may provide the axial driving force to actuate the predefined sequence that the cutting needle (63), and thereby the cutting edge (64), may be urged through the through bore (16) of the cannula (15). The user operable actuator (65) may be sized and shaped to be comfortable and ergonomically located for a user to hold and operate the device for meniscal repair (100) over an extended period of time.

In this particular embodiment, the incremental advancement of the cutting needle (63) and the cutting edge (64) through the through bore (16) of the cannula (15) from its initial position at the distal end (18) of the cannula (15) to the final position at the proximal end (19), may urge the cutting edge (64) to project beyond the proximal end (18) of the cannula (15). Also in this particular embodiment, it will be appreciated that the user operable actuator (65) may provide the means to incrementally retreat the cutting needle (63) and the cutting edge (64) through the through bore (16) of the cannula (15), retreating from its final position at the proximal end (19) of the cannula (15) to its distal end (18). In this way, the user may control either the incremental advancement or retreat of the cutting needle (63) and the cutting edge (64) through the through bore (16) of the cannula (15).

In one embodiment, the actuation of the user operable actuator (65) to incrementally advance the pusher assembly (62) through the through bore (16) of the cannula (15) may provide a tactile and audible indication of the advancement of the cutting needle (63) through the through bore (16) of the cannula (15). In this way, the user is provided with an indication via the tactile and audible response that the user operable actuator (65) is actuated, and that cutting needle (63) and the cutting member (64) have either advanced or retreated relative to the proximal end (19) through the through bore (16) of the cannula (15).

In any one of the above embodiments, whereby the second embodiment of the device for meniscal repair (100) comprises the cutter member (60′), the cutting needle (63) may comprise a flexible end (66) which flexes to an angle of the proximal end (19) of the cannula (15). In this way, when the proximal end (19) of the cannula (15) is set at a particular angle, the flexible end (66) of the cutting needle (63) may flex to said particular angle. It will be appreciated that the flexible end (66) may be sized and shaped to be received within the through bore (16), and project from the proximal end (19) of the cannula (15), whereby it may also comprise the cutting edge (64).

In any one of the above embodiments, the handle (10) of the second embodiment of the device for meniscal repair (100) comprising the cutter member (60), may further comprise an indicating means (54) to provide a visual indication of the incremental advancement of the cutting edge (64) through the through bore (16) of the cannula (15). Particularly with reference to FIG. 10, it is illustrated that the indicating means (54) may be positioned on the handle (10) so as to provide the user with the visual indication.

In this particular embodiment, the indicating means (54) may comprise one or more indicia to provide the visual indication of the incremental advancement of the cutting needle (63) and the cutting edge (64) through the through bore (16) of the cannula (15). In this way, the indicating means (54) may include a number of indicia corresponding to the position of the cutting edge (64) and/or the flexible end (66) of the cutting needle (63) relative to the proximal end (19) of the cannula (15). It will be appreciated that further embodiments of the indicating means (54) are envisaged beyond those discussed, such as any indicating means (54) that may in use provide the user with a visual indication of the position of the cutting edge (64) and/or the flexible end (66) of the cutting needle (63) relative to the proximal end (19) of the cannula (15).

In one embodiment, the cutter member (60 or 60′) may further comprise a third attachment portion (67), co-operable with the first attachment portion (17) of the handle (10 or 20) so as to form an attachment mechanism (not shown) to effect securement of the cutter member (60 or 60′) to the handle (10 or 20). In this way, the cutter member (60 or 60′) is secured to the handle (10 or 20) so as to form the second embodiment of the device for meniscal repair (100).

In one embodiment, one of the handle (10) or the cutter member (60) comprises a user operable release (68) for the attachment mechanism.

In this particular embodiment, one of either the first (17) or third (67) attachment portions comprises a biasing means (not shown) which biases the first (17) or third (67) attachment portions into a capture position with respect to the other attachment portion. The biasing means may bias either one of the first (17) or third (67) attachment portions into a capture position with respect to the other attachment portion. In this way, the capture position may represent a locked position in which the cutter member (60) is secured to the handle (10), thereby forming the second embodiment of the device for meniscal repair (100).

In this particular embodiment, the user operable release (68) may act against the biasing means, thereby releasing one of the first (17) or third (67) attachment portions from the capture position. The biasing means may be manufactured of a resilient material which biases one of the first (17) or third (67) attachment portions into the capture position with respect to the other attachment portion. The biasing means may comprise a number of features such as a spring (not shown) which may be used to bias one of the first (17) or third (67) attachment portions into the capture position with respect to the other attachment portion. Alternate biasing means beyond those described in the embodiments of this disclosure are also envisaged, it will be appreciated by those skilled in the art that the biasing means coupled with the first (17) and third (67) attachment portions are designed so as to provide a releasable mechanism that effects securement of the cutter member (60) to the handle (10), thereby forming the second embodiment of the device for meniscal repair (100). It will be further appreciated that the releasable mechanism that effects this securement may be known to provide for quick coupling and uncoupling of the cutter member (60) from the handle (10), whereby the handle (10) may be further used to receive another member (such as the deployment member (50)) within the socket (14).

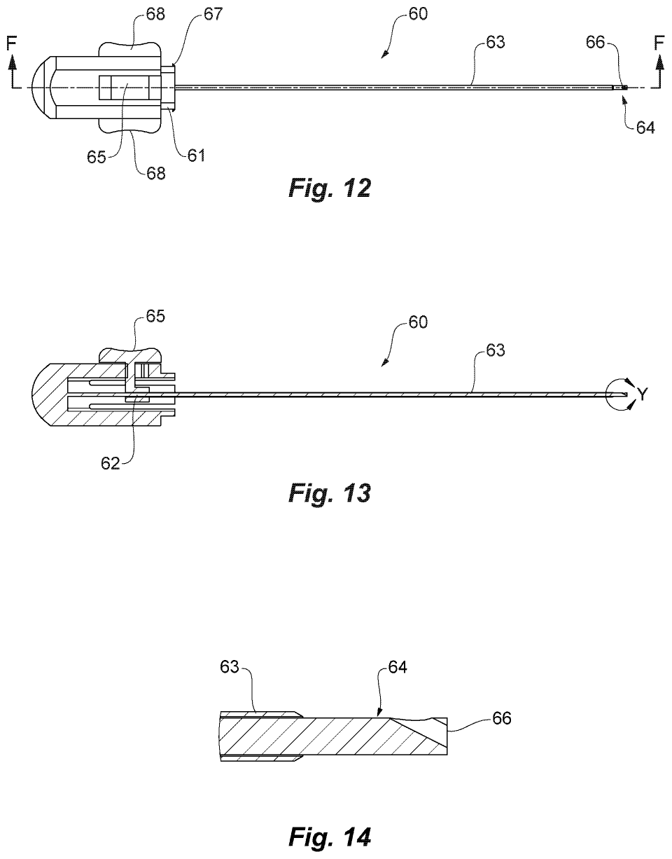

In an alternative embodiment, the user operable release (68) may take the form of a button (not shown) or a trigger (not shown), and may be comprised on either one of the handle (10) or the cutter member (60) of the device for meniscal repair (100). Illustrated in FIG. 12, the user operable release (68) may take the form of two or more buttons (68) that must be actuated simultaneously in order to provide the releasable mechanism that effects securement of the cutter member (60) to the handle (10). It will be appreciated that the user operable release (68) may take further alternate forms beyond those described in these embodiments. In this way, the user operable release (68) provides the releasable mechanism that effects securement of the cutter member (60) to the handle (10).

In another alternative embodiment, with particular reference to FIGS. 31 to 34 and the alternate cutter member (60′), the third (67) attachment portion may be a cantilever snap and the first attachment portion (17) of the alternate handle (20) may be a keeper for the cantilever snap. In this particular embodiment, the user operable release (68) acts against the cantilever snap, thereby releasing it from the keeper. In this way, the user operable release (68) provides the releasable mechanism that effects securement of the alternate cutter member (60′) to the alternate handle (20).

In another alternative embodiment, one of the first (17) or third (67) attachment portions may comprise one or more ribs, and the other may comprise one or more keepers (not shown) corresponding to the one or more ribs. In this particular embodiment, the user operable release (68) may act against the attachment mechanism formed by the cooperation of the one or more ribs with the one or more keepers to provide the releasable mechanism that effects securement of the cutter member (60) to the handle (10).

In a further alternate embodiment, it will be appreciated that the second embodiment of the device for meniscal repair (100) comprising the cutter member (60 or 60′) may be designed without the user operable release (68), or the biasing means. In this embodiment, the cutter member (60 or 60′) may simply be secured to the handle (10 or 20) by virtue of the second projection (61) being sized and shaped to be received within the socket (14). In this way, a person skilled in the art will appreciate that the cutter member (60 or 60′) may be removably secured to the handle (10 or 20).

In one embodiment, the cutting needle (63) of the cutter member (60 or 60′) may be sized and shaped to be received within the through bore (16) of the cannula (15).

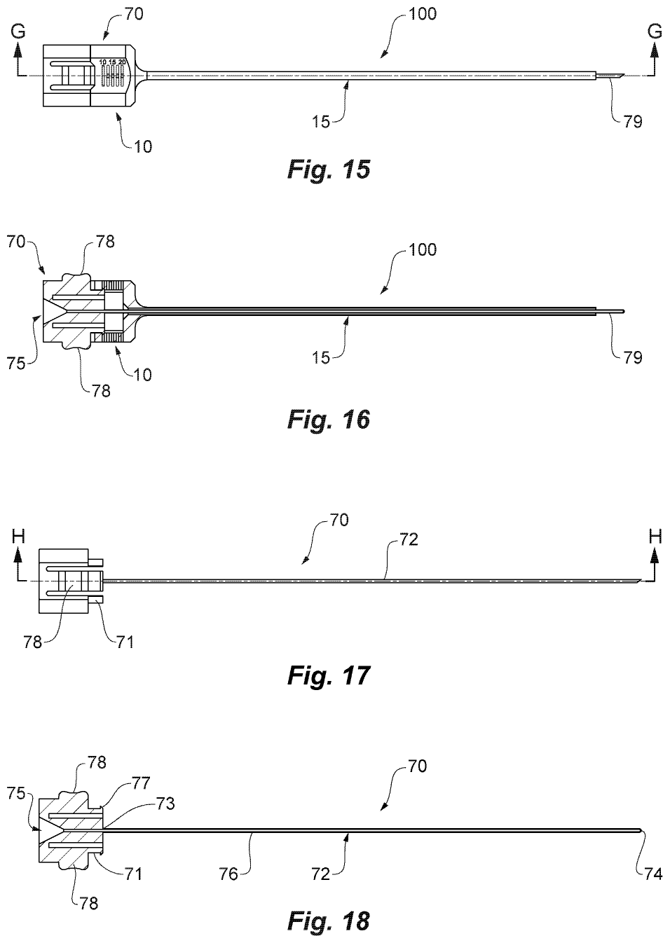

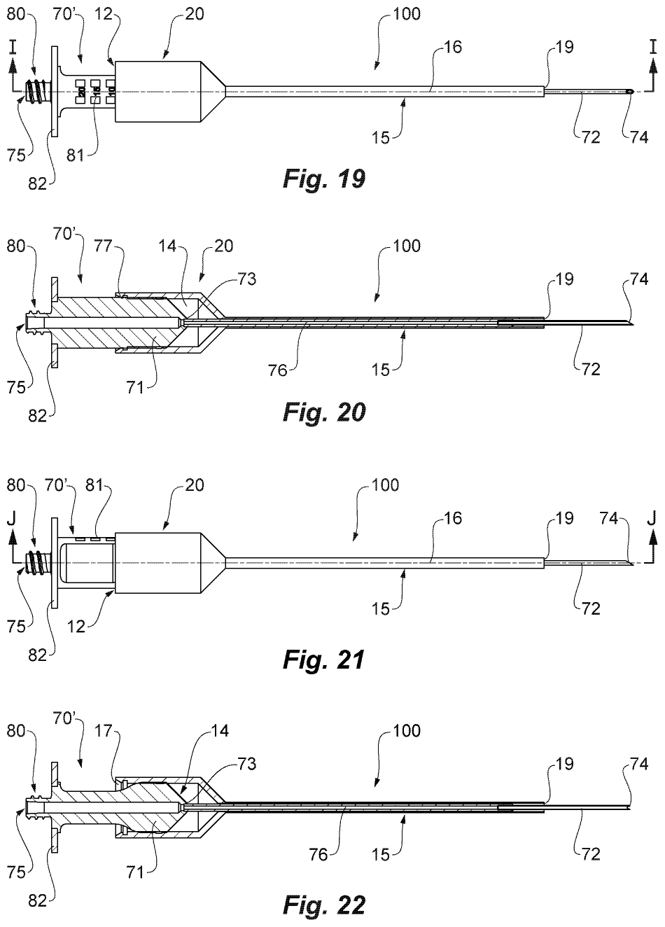

Referring now to any one of FIGS. 15 to 22, in another alternative embodiment the device for meniscal repair (100) may further comprise a fluid injection member (70) (illustrated in FIGS. 15 to 18) and an alternate fluid injection member (70′) (illustrated in FIGS. 19 to 22) for injecting a fluid. The fluid injection members (70 or 70′) may comprise a third projection (71), where the third projection (71) may be sized and shaped to be received within the socket (14) of either the handle (10) (illustrated in FIGS. 15 and 16) or the alternate handle (20) (illustrated in FIGS. 19 to 22). In this way, it will be appreciated, that the socket (14) of the handle (10 or 20) may house the third projection (71) of the fluid injection members (70 or 70′). Thereby, in use, the handle (10 or 20) and the fluid injection members (70 or 70′) cooperate so as to form a third embodiment of the device for meniscal repair (100).

In one embodiment, the fluid injection members (70 or 70′) may comprise a needle (72). Whereby the needle (72) extends from a distal end (73) to a proximal end (74) of the fluid injection member (70).

In one embodiment, the fluid injection members (70 or 70′) may further comprise a second inlet (75). Whereby the second inlet (75) provides for a fluid passageway (76) extending between the second inlet (75) to the proximal end (74) of the needle (72).

In an alternate embodiment to above, the second inlet (75) may be sized and shaped to provide for a fluid passage way (not shown) extending between the second inlet (75) of the fluid injection members (70 or 70′) and co-operable with the through bore (16) of the cannula (15) for expelling the fluid through the proximal end (19) of the cannula (15) via the through bore (16).

In one embodiment, the second inlet (75) is sized and shaped to receive the fluid to be injected through the fluid passageway (76), whereby the fluid is expelled at the proximal end (74) of the needle (72).

In one embodiment, referring in particular to FIGS. 19 to 22, the alternate fluid injection member (70′) may comprise an attachment portion (80) proximal to the second inlet (75). The attachment portion (80) may extend in a direction rearwardly and away from the third projection (71) so as to permit securement of an external device (not shown) to the alternate fluid injection member (70′). In one embodiment, the attachment portion (80) may be threaded, so as to enable securement of the alternate fluid injection member (70′) to the external device. It will be appreciated by those skilled in the art that alternate embodiments of the attachment portion (80) are envisaged beyond that disclosed herein, such that the attachment portion (80) enables the securement of the alternate fluid injection member (70′) to the external device. It will further be appreciated by those skilled in the art, that the external device may be any one of a fluid container, a fluid reservoir, or another source of fluid to be injected through the alternate fluid injection member (70′).

In one embodiment, still referring to FIGS. 19 to 22, the alternate injection member (70′) may further comprise a depth indicating means (81) to provide a visual indication of a depth to which the proximal end (74) of the needle (72) is relative to the proximal end (19) of the cannula (15). In this way, the depth indicating means (81) provides the user with a visual indication of the depth to which the proximal end (74) of the needle (72) is inserted into the tissue/meniscus.

In one embodiment, still referring to FIGS. 19 to 22, the alternate injection member (70′) may further comprise a flange (82) proximal to the second inlet (75) and extending from the third projection (71). The flange (82) being sized and shaped so as to abut against the first inlet (12) of the alternative handle (20), and in this way, limit the depth that the proximal end (74) of the needle (72) may be inserted into the tissue/meniscus.

In one embodiment, the fluid may be a biological agent or an adhesive or a local anaesthetic or an adjuvant agent used for meniscal repair. In this way, the fluid may be selected so as to improve the biological condition for the tissue/meniscus to heal. It will be appreciated that the fluid may also be selected for various other applications, beyond that discussed of being selected for the purposes of further aiding in the healing process of repairing a torn tissue/meniscus. In this way, the fluid injection members (70 or 70′) may be referred to as biological agent delivery members (70 or 70′).

In one embodiment, the fluid injection members (70 or 70′) may further comprise a fourth attachment portion (77), co-operable with the first attachment portion (17) of the handle (10 or 20) so as to form an attachment mechanism (not shown) to effect securement of the fluid injection members (70 or 70′) to the handle (10 or 20). In this way, the fluid injection members (70 or 70′) are secured to the handle (10 or 20) so as to form the third embodiment of the device for meniscal repair (100).

In one embodiment, one of the handle (10) or the fluid injection member (70) comprises a user operable release (78) for the attachment mechanism.

In this particular embodiment, one of the first (17) or fourth (77) attachment portions comprises a biasing means (not shown) which biases the first (17) or fourth (77) attachment portions into a capture position with respect to the other attachment portion. The biasing means may bias either one of the first (17) or fourth (77) attachment portions into a capture position with respect to the other attachment portion. In this way, the capture position may represent a locked position in which the fluid injection member (70) is secured to the handle (10), thereby forming the third embodiment of the device for meniscal repair (100).

In this particular embodiment, the user operable release (78) may act against the biasing means, thereby releasing one of the first (17) or fourth (77) attachment portions from the capture position. The biasing means may be manufactured of a resilient material which biases one of the first (17) or fourth (77) attachment portions into the capture position with respect to the other attachment portion. The biasing means may comprise a number of features such as a spring (not shown) which may be used to bias one of the first (17) or fourth (77) attachment portions into the capture position with respect to the other attachment portion. Alternate biasing means beyond those described in the embodiments of this discloser are also envisaged, it will be appreciated by those skilled in the art that the biasing means coupled with the first (17) and fourth (77) attachment portions are designed so as to provide a releasable mechanism that effects securement of the fluid injection member (70) to the handle (10), thereby forming the third embodiment of the device for meniscal repair (100). It will be further appreciated that the releasable mechanism that effects this securement, may be known to provide for quick coupling and uncoupling of the fluid injection member (70) from the handle (10), whereby the handle (10) may be further used to receive a another member (such as either the deployment member (50, or 50′) or the cutter member (60, or 60′)) within the socket (14).

In an alternative embodiment, the user operable release (78) may take the form of a button (not shown) or a trigger (not shown), and may be comprised on either one of the handle (10) or the fluid injection member (70) of the device for meniscal repair (100). Illustrated in any one of FIG. 16 or 18, the user operable release (78) may take the form of two or more buttons (78) that must be actuated simultaneously in order to provide the releasable mechanism that effects securement of the fluid injection member (70) to the handle (10). It will be appreciated that the user operable release (78) may take further alternate forms beyond those described in these embodiments. In this way, the user operable release (78) provides the releasable mechanism that effects securement of the fluid injection member (70) to the handle (10).

In another alternative embodiment, one of the first (17) or fourth (77) attachment portions may be a cantilever snap, and the other may be a keeper for the cantilever snap. In this particular embodiment, the user operable release (78) acts against the cantilever snap, thereby releasing it from the keeper. In this way, the user operable release (78) provides the releasable mechanism that effects securement of the fluid injection members (70 or 70′) to the handle (10 or 20).

In another alternative embodiment, one of the first (17) or fourth (77) attachment portions may comprise one or more ribs, and the other may comprise one or more keepers (not shown) corresponding to the one or more ribs. In this particular embodiment, the user operable release (78) may act against the attachment mechanism formed by the cooperation of the one or more ribs with the one or more keepers to provide the releasable mechanism that effects securement of the fluid injection members (70 or 70′) to the handle (10 or 20).

In a further alternate embodiment, it will be appreciated that the third embodiment of the device for meniscal repair (100) comprising the fluid injection members (70 or 70′) may be designed without the user operable release (78), or the biasing means. In this embodiment, the fluid injection members (70 or 70′) may simply be secured to the handle (10 or 20) by virtue of the third projection (71) being sized and shaped to be received within the socket (14). In this way, a person skilled in the art will appreciate that the fluid injection members (70 or 70′) may be removably secured to the handle (10 or 20).

In one embodiment, the needle (72) may be sized and shaped to be received within the through bore (16) of the cannula (15).

In one embodiment, a projected portion (79) of the needle (72) may extend from the proximal end of the cannula (15).

In one embodiment, referring still to the third embodiment of the device for meniscal repair (100) comprising the fluid injection members (70 or 70′), the proximal end (74) of the needle (72) is flexible and flexes to an angle of the proximal end of the cannula (15).

In the above embodiment, it will be appreciated that a proximal end (19) of the cannula (15) may be at an angle between 0° and 90°. Referring now to FIG. 3, there are illustrated five embodiments of the handle (10), with the proximal end (19) of the cannula (15) at angles of 0° (illustrated as handle 10), 20° (illustrated as handle 10′), 40° (illustrated as handle 10″), 60° (illustrated as handle 10′″) and 85° (illustrated as handle 10″″) by way of example only. It will be further appreciated that the angle of the cannula (15) may be selected based on the location, complexity, and requirements of the repair in order to access the location of the torn tissue/meniscus.

In any one of the above embodiments, particularly those with reference to the third embodiment of the device for meniscal repair comprising the fluid injection members (70 or 70′), this third embodiment may be used for the repair of soft tissues, such as a meniscus, that may have been torn. The repair of the tissue/meniscus may be carried out by known arthroscopic or semi-open procedures well known, utilising the embodiments of the device for meniscal repair (100) by the creation of at least one incision to a patient created by insertion of the proximal end of the cannula (15) at the desired incision location. Following the creation of the incision, the cannula (15) of the device for meniscal repair (100) may be surgically directed toward the location repair is to be carried out, and the user of the device (100) may via the second inlet (75), the fluid passageway (76), and the proximal end (74) of the needle (72) deliver the fluid required for the repair. It will be appreciated, that this device (100) comprising either of the fluid injection members (70 or 70′) is the third embodiment (as illustrated by FIGS. 15 to 22) of the device (100) to repair the tissue/meniscus.

In any one of the above embodiments, referring to any one of the first, second or third embodiments of the device for meniscal repair (100), it will be appreciated that the angle at which the proximal end (19) of the cannula (15) of the handle (10) that is selected for use with either the first, second or third embodiments of the device for meniscal repair (100), any one of the flexible end (59) of the deployment member (50′), or the flexible end (66) of the cutter member (60 or 60′), or the projected portion (79) of the fluid injection member (70 or 70′), may flex to match the angle of the proximal end (19) of the cannula (15).

In an alternative embodiment, the proximal end (19) of the cannula (15) may also be flexible. In this way, advantageously the proximal end (19) of the cannula (15) may be flexed to angles between 0° and 90°, as required by the user for better directing the device for meniscal repair (100) toward the tear in the tissue/meniscus. It will be appreciated that in this embodiment, that any one of the flexible end (59) of the deployment member (50 or 50′), or the flexible end (66) of the cutter member (60 or 60′), or the projected portion (79) of the fluid injection member (70 or 70′), may flex to match the angle of the proximal end (19) of the cannula (15).

In any one of the above embodiments, still referring to any one of the first, second or third embodiments of the device for meniscal repair (100), it will be appreciated that the flexibility of any one of the flexible end (59) of the deployment member (50 or 50′), or the flexible end (66) of the cutter member (60 or 60′), or the projected portion (79) of the fluid injection member (70 or 70′), or the proximal end (19) of the cannula (15), advantageously permits better direction of the device for meniscal repair (100) toward the tear in the tissue/meniscus for repair.

In any one of the above embodiments, it will be appreciated by those skilled in the art that the cooperation of the first attachment portion of the handle (10 or 20), the second attachment portion of the deployment member (50 or 50′), or the third attachment portion (67) of the cutter member (60 or 60′), or the fourth attachment portion (77) of the fluid injection member (70 or 70′), the biasing means and the user operable releases (any one of 56, 68, 78) are intended to provide the device for meniscal repair (100) with the attachment mechanism. Thereby allowing the user to readily and easily switch between the deployment member (50 or 50′), the cutter member (60 or 60′) and the fluid injection member (70 or 70′) for use with the handle (10 or 20), thereby performing a repair to a torn tissue/meniscus. The attachment mechanism of the device (100) may be referred to as a universal clip arrangement, to advantageously provide the ease of switching between the deployment member (50 or 50′), the cutter member (60 or 60′) and the fluid injection member (70 or 70′). It will also be appreciated that the handle (10 or 20) comprising the first attachment portion is capable of forming the universal clip arrangement (attachment mechanism) and may be used with a variety of other devices used in surgical procedure, whereby the other device to be used comprises the second attachment portion. It will further be appreciated that advantageously the first, second and third embodiments of the device for meniscal repair (100) permits the quick coupling and uncoupling of any one of the deployment member (50 or 50′), the cutter member (60 or 60′) or the fluid injection member (70 or 70′) from the handle (10 or 20), whereby the handle (10 or 20) may subsequently be further used to receive a another member (such as either the deployment member (50 or 50′), the cutter member (60 or 60′), or the fluid injection member (70 or 70′)) within the socket (14).

In any one of the above embodiments, referring still to any one of the first, second or third embodiments of the device for meniscal repair (100), the indicating means (54) may further indicate the securement effect of any one of the deployment member (50), cutter member (60) or the fluid injection member (70) to the handle (10) by one or more indicia indicating that the first attachment portion (17) of the handle (10) has engaged the second (55), third (67) or fourth (77) attachment portions, so as to cooperate to form the universal clip arrangement (attachment mechanism). In this way, the user may confirm via the indicating means (54) that any one of the deployment member (50), cutting member (60) or the fluid injection member (70) is secured to the handle (10).

In one embodiment, the device for meniscal repair (100) of any one of the first, second or third embodiments may further comprise a depth gauge (not shown). Whereby the depth gauge is particularly designed so as to limit the depth that either the cannula (15) may be inserted into the tissue/meniscus.

The cannula (15) of any one of the above embodiments may further comprise either laser marks (not shown) or coloured bars (not shown) along a length of any one of the deployment needle (58) of the deployment member (50 or 50′), or the cutting needle (63) of the cutter member (60 or 60′), or the needle (72) of the fluid injection member (70 or 70′), from their respective distal ends to their respective proximal ends. The laser marks or the coloured bars along the lengths of any one of the deployment needle (58) of the deployment member (50 or 50′), or the cutting needle (63) of the cutter member (60 or 60′), or the needle (72) of the fluid injection member (70 or 70′), may be configured so as to provide the user (during use of the device for meniscal repair (100)) an indication of a depth to which any one of these needles (58, 63 or 72) are inserted into the tissue/meniscus. It will be appreciated that, in this way, the cannula (15) creates an incision and directs the device for meniscal repair (100) toward the repair area of the tissue/meniscus, and subsequently any one of the needles (58, 63 or 72) may perform their function in the repair of the tissue/meniscus whilst advantageously the laser marks or the coloured bars provide the user with an indication of the depth at which the needle (58, 63 or 72) is inserted into the tissue/meniscus. In an alternative embodiment, the laser marks or the coloured bars on any one of the needles (58, 63 or 72) may be located along a length of a proximal end of each of the needles (58, 63 or 72). In this way, the laser marks or the coloured bars on any one of the needles (58, 63 or 72) may advantageously allow the user to more accurately perform the functions of any one of the first, second or third embodiments of the device for meniscal repair (100) to repair the tissue/meniscus.

In addition to the above, the cannula (15) and any one of the deployment needle (58) of the deployment member (50 or 50′), or the cutting needle (63) of the cutter member (60 or 60′), or the needle (72) of the fluid injection member (70 or 70′) may be manufactured of a translucent or a transparent material. In this way:
the one or more anchors deployed by the deployment needle (58) are visible through both the deployment needle (58) and the cannula (15), so as to advantageously permit the user to visually identify and confirm deployment of the one or more anchors via the deployment needle (58);
progress of the cutting edge (64) urged through the cannula (15) by the cutting needle (63) is visible through both the cutting needle (63) and the cannula (15), so as to advantageously permit the user to visually identify and confirm the position and progress of the cutting edge (64) through the cutting needle (63) and the cannula (15); and
the position of the needle (72) is visible through the cannula (15), so as to advantageously permit the user to visually identify and confirm the position of the needle (72) through the cannula (15).

Thus it will be appreciated that the device for meniscal repair (100) of any of the above embodiments may be utilised as a surgical device for the repair of one or more tears in a meniscus or tissue.

In any one of the above embodiments of the device for meniscal repair (100), it will be appreciated that the device (100) may take either one of the first (illustrated by FIGS. 1, 2, 7, 8 and 23 to 26) or the second (illustrated by FIGS. 10 to 14, and 31 to 34), or third (illustrated by FIGS. 15 to 22) forms. In this way, the device for meniscal repair (100) advantageously provides functionality that would normally require more than one known medical devices for the surgical repair of a tissue/meniscus. It will be further appreciated, that in use, the device for meniscal repair (100) may permit the user to perform a number of surgical actions required for the repair of a tear in a tissue/meniscus. In this way, the device (100) may be considered a system for meniscal repair, for example:
a. As a first step, the device (100) comprising only the handle (10 or 20) (referring to any one of FIGS. 3 to 6), may be used to create an incision to a tissue requiring repair by the insertion of the proximal end (19) of the cannula (15) at the desired location on a patient.
b. As a second step, the deployment member (50 or 50′) (referring to any one of FIGS. 1, 2, 7, 8 and 23 to 26) may have its first projection (51) received within the socket (14) of the handle (10 or 20), whereby the first (17) and second (55) attachment portions co-operate to form the attachment mechanism, allowing the user to actuate the user operable actuator (53) to incrementally advance the injector assembly (52), and thus the deployment needle (58) through the through bore (16) of the cannula (15) to deliver the desired number of anchors required to repair the tear, whilst the user receives confirmation of the deployment of the corresponding anchor at the tear via the tactile and audible response, and a visual indication of the deployment via the indicating means (54).
c. As an intermediary step, once the use of the deployment member (50 or 50′) is finished, the user operable release (56) may be actuated to release the deployment member (50 or 50′) from the handle (10 or 20), thereby allowing the user to remove the deployment member (50 or 50′), whilst the handle (10 or 20) and thus the cannula (15) remains at the location in the tissue requiring repair.
d. As a third step, the cutter member (60 or 60′) may have its second projection received within the socket (14) of the handle (10 or 20), whereby the first (17) and third (67) attachment portions co-operate to form the attachment mechanism, allowing the user to actuate the user operable actuator (65) to manipulate, cut, tension, slacken or remove suture required for the repair, whilst the user receives confirmation that the cutting needle (63) and the cutting edge (64) have either advanced or retreated relative to the proximal end (19) through the through bore (16) of the cannula (15) at the tear via the tactile and audible response, and a visual indication via the indicating means (54).
e. As an additional intermediary step, once the use of the cutter member (60 or 60′) is finished, the user operable release (68) may be actuated to release the cutter member (60 or 60′) from the handle (10 or 20), thereby allowing the user to remove the cutter member (60 or 60′), whilst the handle (10 or 20) and thus the cannula (15) continues to remain at the location in the tissue requiring repair.
f. As a fourth step, the fluid injection member (70 or 70′) may have its third projection received within the socket (14) of the handle (10 or 20), whereby the first (17) and fourth (77) attachment portions co-operate to form the attachment mechanism, allowing the user to inject a fluid to the location for repair to aid in improving the biological condition for the tissue/meniscus to heal.
g. Subsequently, as a final step, the device for meniscal repair (100) comprising the fluid injection member (70 or 70′) may be removed from the repaired tissue (and thus the incision in the patient), thereby finishing the repair process. That is to say both the injection member (70 or 70′) and the handle (10 or 20′) to which it is secured are removed from the repaired tissue. Alternatively, if subsequent anchors, suture or fluid is required, subsequent uses of additional deployment members (50 or 50′), cutter members (60 or 60′) and/or fluid injection members (70 or 70′) may be utilised with the handle (10 or 20) and above steps (a) to (f) may be repeated as required.

Thus, in the above example provided, the system for meniscal repair (100) advantageously permits the user to target the tear in the tissue for repair, deploy anchors, manipulate/tension/slacken/cut lengths of suture, and apply the fluid to aid in improving the biological conditions for the tissue to heal, without the requirement of another device. An additional advantage of the system (100) is in that there is reduced risk for the user to erroneously inject fluid at a location other than the location of that the anchors were deployed, and subsequently increases the likelihood of successful repair to the tissue.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope of the disclosure as set forth and defined by the following claims.

1. A device for meniscal repair comprising:
a handle comprising an axial bore extending between a first inlet and an outlet, and a first attachment portion;
a cannula coupled to the outlet, wherein the cannula comprises a through bore cooperative with the axial bore of the handle, and a proximal end that is at an angle between 0° and 90°; and
wherein a portion of the axial bore at the first inlet of the handle is sized and shaped to form a socket, wherein the socket provides access to the axial bore of the handle and the through bore of the cannula.

2. The device for meniscal repair of claim 1, further comprising a deployment member, wherein the deployment member comprises a first projection sized and shaped to be received within the socket of the handle, an injector assembly configured to incrementally advance through the through bore of the cannula from a distal end to the proximal end of the cannula, wherein the injector assembly comprises one or more anchors, wherein the anchors are urged through the through bore in a predefined sequence to the proximal end, and a user operable actuator coupled to the injector assembly, wherein the user operable actuator provides an axial driving force to actuate the injector assembly.

3. (canceled)

4. The device for meniscal repair of claim 2, wherein the incremental advancement of the injector assembly through the through bore of the cannula provides a deployment of an anchor of the one or more anchors from the proximal end of the cannula.

5. The device for meniscal repair of claim 2, wherein the actuation of the user operable actuator to incrementally advance the injector assembly through the through bore of the cannula provides a tactile and audible indication of a deployment of the corresponding anchor of the one or more anchors.

6. The device for meniscal repair of claim 2, wherein the deployment member further comprises a deployment needle sized and shaped to be received within the through bore of the cannula, wherein the deployment needle comprises a flexible end which flexes to the angle of the proximal end of the cannula, and whereby the deployment needle deploys the anchor of the one or more anchors at the proximal end of the cannula.

7. (canceled)

8. The device for meniscal repair of claim 2, wherein one of the handle or the deployment member further comprises an indicating means to provide a visual indication of a deployment of the corresponding anchor.

9. The device for meniscal repair of claim 2, wherein the deployment member further comprises a second attachment portion co-operable with the first attachment portion to form an attachment mechanism to effect securement of the deployment member to the handle, and wherein one of the handle or the deployment member comprises a user operable release for the attachment mechanism.

10. (canceled)

11. The device for meniscal repair of claim 9, wherein at least one of the first or second attachment portions comprises a biasing means which biases the first or second attachment portions into a capture position with respect to the other attachment portion, and wherein the user operable release acts against the biasing means, thereby releasing one of the first or second attachment portions from the capture position.

12. (canceled)

13. The device for meniscal repair of claim 1, further comprising a cutter member, wherein the cutter member comprises a second projection sized and shaped to be received within the socket of the handle, a pusher assembly configured to incrementally advance a cutting needle through the through bore of the cannula from a distal end to the proximal end of the cannula, wherein a cutting edge of the cutting needle is urged through the through bore in a predefined sequence to the proximal end, a user operable actuator coupled to the pusher assembly, wherein the user operable actuator provides an axial driving force to actuate the pusher assembly, and wherein the cutting needle comprises a flexible end which flexes to the angle of the proximal end of the cannula.

14. The device for meniscal repair of claim 13, wherein the incremental advancement of the pusher assembly through the through bore of the cannula progressively protrudes the cutting edge of the cutting needle through the proximal end of the cannula.

15. The device for meniscal repair of claim 13, wherein the actuation of the user operable actuator to incrementally advance the pusher assembly through the through bore of the cannula provides a tactile and audible indication of the advancement of the cutting needle through the through bore of the cannula.

16. (canceled)

17. The device for meniscal repair of claim 13, wherein one of the handle or the cutter member further comprises an indicating means to provide a visual indication of the incremental advancement of the cutting edge through the through bore of the cannula.

18. The device for meniscal repair of claim 13, wherein the cutter member further comprises a third attachment portion co-operable with the first attachment portion to form an attachment mechanism to effect securement of the cutter member to the handle, and wherein one of the handle or the cutter member comprises a user operable release for the attachment mechanism.

19. (canceled)

20. The device for meniscal repair of claim 17, wherein at least one of the first or third attachment portions comprises a biasing means which biases the first or third attachment portions into a capture position with respect to the other attachment portion, and wherein the user operable release acts against the biasing means, thereby releasing one of the first or third attachment portions from the capture position.

21. (canceled)

22. (canceled)

23. The device for meniscal repair of claim 1, further comprising a fluid injection member for injecting a fluid, wherein the fluid injection member comprises a third projection sized and shaped to be received within the socket of the handle, a needle extending from a distal end to a proximal end of the fluid injection member, a second inlet providing a fluid passageway extending between the second inlet to a proximal end of the needle, and wherein the proximal end of the needle is flexible and flexes to the angle of the proximal end of the cannula.

24. The device for meniscal repair of claim 23, wherein the second inlet is sized and shaped to receive the fluid to be injected through the fluid passageway, whereby the fluid is expelled at the proximal end of the needle.

25. (canceled)

26. The device for meniscal repair of claim 23, wherein the fluid injection member further comprises a fourth attachment portion co-operable with the first attachment portion to form an attachment mechanism to effect securement of the fluid injection member to the handle, and wherein one of the handle or the fluid injection member comprises a user operable release for the attachment mechanism.

27-34. (canceled)

35. The device for meniscal repair of claim 1, wherein the cannula is manufactured of a transparent or a translucent material.

36. A device for meniscal repair comprising:
a handle comprising an axial bore extending between a first inlet and an outlet, and a first attachment portion;
a cannula coupled to the outlet, wherein the cannula comprises a through bore cooperative with the axial bore of the handle, and a proximal end that is at an angle between 0° and 90°;
wherein a portion of the axial bore at the first inlet of the handle is sized and shaped to form a socket, wherein the socket provides access to the axial bore of the handle and the through bore of the cannula;
and a deployment member comprising a first projection sized and shaped to be received within the socket of the handle, a deployment needle sized and shaped to be received within the through bore comprising a flexible end which flexes to the angle of the proximal end, an injector assembly configured to incrementally advance through the through bore of the cannula from a distal end to the proximal end, a user operable actuator coupled to the injector assembly, wherein the user operable actuator provides an axial driving force to actuate the injector assembly, and a second attachment portion co-operable with the first attachment portion to form an attachment mechanism to effect securement of the deployment member to the handle.

37.-44. (canceled)

45. A device for meniscal repair comprising:
a handle comprising an axial bore extending between a first inlet and an outlet, and a first attachment portion;
a cannula coupled to the outlet, wherein the cannula comprises a through bore cooperative with the axial bore of the handle, and a proximal end that is at an angle between 0° and 90°;
wherein a portion of the axial bore at the first inlet of the handle is sized and shaped to form a socket, wherein the socket provides access to the axial bore of the handle and the through bore of the cannula; and
a cutter member, wherein the cutter member comprises a second projection sized and shaped to be received within the socket of the handle, a pusher assembly configured to incrementally advance a cutting needle through the through bore of the cannula from a distal end to the proximal end, wherein a cutting edge of the cutting needle is urged through the through bore in a predefined sequence to the proximal end, a user operable actuator coupled to the pusher assembly, the cutting needle comprising a flexible end which flexes to the angle of the proximal end, wherein the user operable actuator provides an axial driving force to actuate the pusher assembly, and a third attachment portion co-operable with the first attachment portion to form an attachment mechanism to effect securement of the cutter member to the handle.

46.-59. (canceled)
